(19) United States
(12) Patent Application Publication
(10) Pub. No.: US 2012/0116401 A1
(43) Pub. Date: May 10, 2012

(54) REPOSITIONING DEVICE FOR THE HANDLING OF DISTAL RADIUS FRACTURES BY MEANS OF A T-SHAPED OSTEOSYNTHESIS PLATE

(76) Inventors: Georg Gradl, Boergerende (DE); Hans-Werner Stedtfeld, Rostock (DE)

(21) Appl. No.: 13/380,133

(22) PCT Filed: Jun. 23, 2010
(86) PCT No.: PCT/EP10/58910

§ 371 Date: Dec. 22, 2011

(30) Foreign Application Priority Data
Jun. 25, 2009 (DE) 102009030622.6

(51) Int. Cl. A61B 17/80

(52) U.S. Cl. 606/70

(57) ABSTRACT

The invention relates to a repositioning device for the handling of distal radius fractures by means of a T-shaped osteosynthesis plate, comprising a long leg and a short leg, disposed in a T-shaped and angular manner in relation to said long leg, wherein the short, distal leg has a face that comes into contact with the distal fracture fragment and a number of openings for bone screws and the long, proximal leg is intended for making contact with the proximal bone near the body. To improve the repositioning device in such a way that the required tilting of the articulated face in the palmar direction of the osteosynthesis plate used can be carried out in an angularly faithful manner, the lower face (14) of the long, proximal leg (13) of the T-shaped plate (7) that is facing the proximal bone or radial shall (11) is provided with a spacer (15), in order to keep the long leg (13) at a certain angle (γ) in relation to the proximal bone (11) near the body.

The invention relates to a repositioning device and a method for the treatment of distal radius fractures by means of a T-shaped osteosynthesis plate according to the preamble of claim 1.

The use of angle-stable plates is known for the treatment of fractures of the forearm distant from the body (spoke bone, radius). A problem here is to obtain an adequate repositioning of the fracture fragments. The outcome should be to restore the human anatomy before the fall.

The anatomical position in the frontal plane with an angle of 25° steepness and approx. 1-5 mm additional length with respect to the ulna at the level of the distal radioulnar joint is decisive for the distal spoke bone (radius) distant from the body. In the sagittal plane, the articular surface of the radius is tilted by approx. 10° in the palmar direction (palm of the hand side). In a typical fracture of the radius, there is a dislocation of the distal fracture fragment out of this anatomical position of around 20-50° in the direction of the back of the hand (dorsal). This dislocation has to be corrected by the treating surgeon. However, this is usually not completely successful. An angle of 0° to 5°, in many cases only a repositioning angle of 0°, is achieved with a conventional operation. In this case, the distal fracture fragment in the sagittal plane is fixed only vertical to the radius shaft.

A T-shaped osteosynthesis plate is previously known from U.S. 2005/0065522 A1. When it is used for an osteotomy, the short leg is screwed by means of bone screws to the distal end of the bone, the subsequent fracture segment, the long leg standing away at an angle from the proximal bone near the body. The bone proximal to the distal end is then sawn off and the plate is swivelled through the angle by means of its long leg to the bone near the body and screwed to the proximal bone near the body by means of bone screws introduced through the long leg into the proximal bone near the body.

The known T-shaped osteosynthesis plate can be used not only in connection with an osteotomy, but also in connection with bone fractures, as has been described at the outset. In the case of a typical fracture of the distal radius distant from the body, the dislocation of the distal fracture fragment has to be corrected by the treating surgeon. However, this is not usually completely successful with the known T-shaped osteosynthesis plate.

The problem underlying the invention, therefore, is to improve the repositioning device and the method for the treatment of distal radius fractures by means of a T-shaped osteosynthesis plate, in such a way that the required tilting of the articular surface in the palmar direction of the employed osteosynthesis plate can be carried out with angular precision.

In order to solve this problem, the invention provides a repositioning device, wherein the lower surface of the long, proximal leg of the T-shaped plate facing the proximal bone or radius shaft is provided with a spacer in order to keep the long leg at a specific angle with respect to the proximal bone near the body.

For this purpose, the spacer beneath the long leg of the T-shaped plate is adjusted to achieve the required angle. When the long leg is then placed with its lower surface, with the interposition of the spacer, on the proximal bone near the body, the short leg of the T-shaped plate already lying with its underside on the dislocated fracture fragment, the T-shaped plate is first fixed distally with the bone screws. The long leg of the T-shaped plate does not therefore lie directly on the proximal bone, but rather stands several millimetres away. An angle between 5° and 20° thus arises between the distal fracture fragment fixed in a neutral position (neutral with respect to the shaft axis of the bone) and the T-shaped plate. If the spacer is now removed by the surgeon and the T-shaped plate is pressed with the long leg onto the proximal bone, the distal fracture fragment fastened to the T-shaped plate is tilted in the palmar direction by the angle of the previously introduced spacer. The anatomy of the distal radius is thus restored in a straightforward manner with a 10° palmar tilt in the sagittal plane, i.e. the remaining 10° tilt of the articular surface in the palmar direction is achieved again together with the T-shaped plate.

In a further embodiment of the invention, the spacer is a distancing element in the form of a wedge, cylinder or parallelepiped that can be disposed on the lower surface of the long leg facing the proximal bone near the body. These distancing elements have to be adapted in their size to the desired angle before insertion in order to achieve a specific, desired angle between the long leg and the proximal bone.

According to the invention, the wedge has an angle of 5° to 20° between its wedge surfaces resting on the proximal bone near the body and on the lower surface of the long leg facing said proximal bone. Furthermore, the wedge can be fastened detachably on the contact side of the long leg facing the proximal bone near the body. With the spacer according to the invention, in this case a wedge, an anatomical repositioning of at least approx. 15° is achieved.

In a further embodiment of the invention, the spacer or the distancing element is a distancing screw that can be screwed in the long leg, said distancing screw comprising a contact face for the proximal bone near the body. Furthermore, the distancing screw can comprise a head thread and a smooth shank. The distancing screw that can be screwed in the long leg enables easy adaptation of the angle between the long leg and the proximal bone to the requirements of the individual application.

The invention also relates to a method for the treatment of distal radius fractures by means of the T-shaped osteosynthesis plate of the repositioning device. The short, distal leg of the T-shaped plate is first screwed to the distal fracture fragment, the long, proximal leg of the T-shaped plate being held spaced apart by means of the spacer at a specific angle with respect to the proximal bone near the body, and finally after removal of the spacer the long leg of the T-shaped plate is pressed onto the proximal bone near the body and is fastened there, as a result of which the distal fracture fragment fastened to the T-shaped plate is tilted by the adjusted angle in the palmar direction.

The repositioning device according to the invention for the treatment of distal radius fractures by means of a T-shaped osteosynthesis plate is explained in greater detail below with the aid of several embodiments. In the figures:

FIG. 1a shows the distal radius of a distal radioulnar joint in the frontal plane,

FIG. 1b shows the distal radius according to FIG. 1a in the sagittal plane,

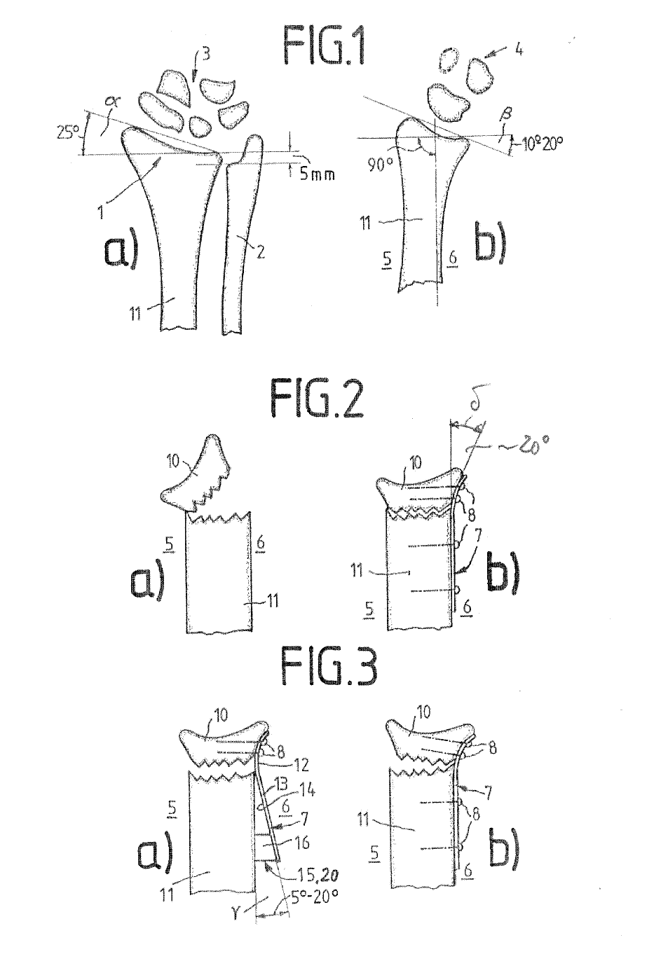

FIG. 2a shows a distal radius with dislocation of the distal fracture fragment in the dorsal direction,

FIG. 2b shows the distal radius fracture according to FIG. 2a post-operatively with a T-shaped osteosynthesis plate according to the prior art,

FIG. 3a shows the distal radius fracture according to FIG. 2a with a T-shaped osteosynthesis plate and with a spacer in the form of a wedge under the long leg of the T-shaped osteosynthesis plate according to the first embodiment of the invention,

FIG. 3b shows the distal radius fracture according to FIG. 3a post-operatively after the removal of the wedge under the long leg of the T-shaped osteosynthesis plate in the first embodiment of the invention,

FIG. 4a shows the second embodiment of the invention, modified from the first embodiment according to FIG. 3a, with a screwed wedge under the long leg of the T-shaped plate,

FIG. 4b shows the fourth embodiment of the invention, modified from the third embodiment according to FIG. 4a, with a distancing screw as a spacer and

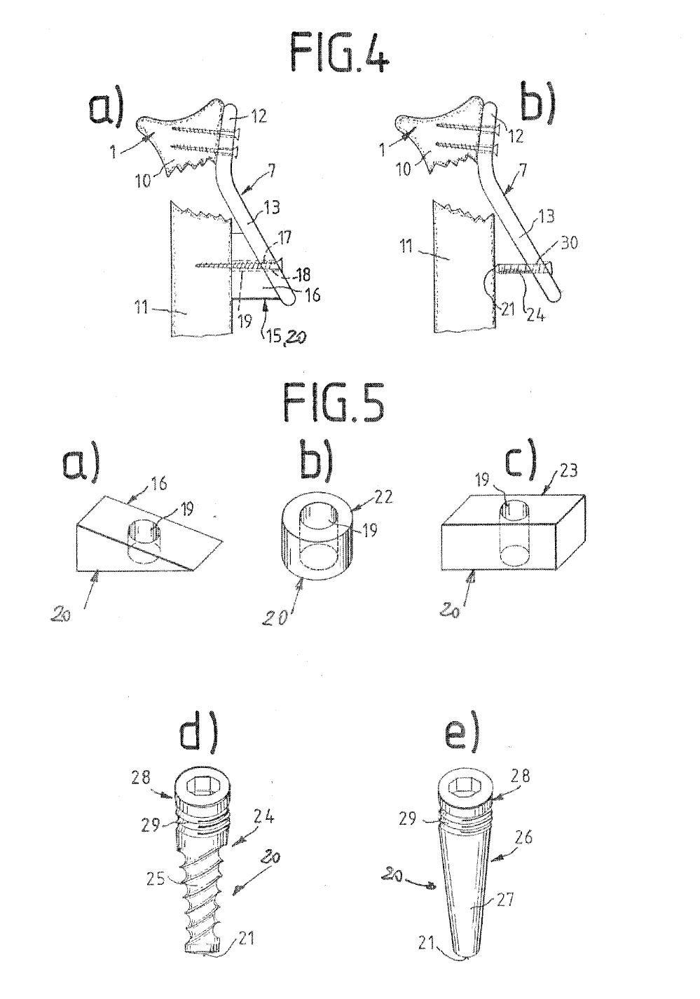

FIG. 5a toe show further embodiments of spacers in the form of a wedge, a cylinder, a parallelepiped, a distancing screw and a screw pin.

FIG. 1a and 1b show a patient's forearm facing outwards from the body (spoke bone, radius) with radius 1, ulna 2, metacarpus 3 and metacarpal bone 4. The problem in the treatment of a fracture of the forearm distant from the body (FIG. 2a) is to achieve adequate repositioning of fracture fragments 10. The aim is to restore the human anatomy before a fall (FIG. 1a, 1b) after a fracture (FIG. 2a). The anatomical position in the frontal plane (FIG. 1a) with an angle α of 25° steepness and approx. 1-5 mm additional length (FIG. 1a) with respect to ulna 2 at the level of the distal radioulnar joint is decisive for distal spoke bone or radius 1 distant from the body. In the sagittal plane according to FIG. 1b, the articular surface of radius 1 is tilted by an angle β of approx. 10° in the paimar direction (palm of the hand side) according to reference number 6 in FIG. 1b and 2a. In a typical fracture of radius 1, there is a dislocation of distal fracture fragments 10 out of this anatomical position by 20 to 50° in the direction of the back of the hand (dorsal) according to reference number 5 in FIG. 2a. This dislocation has to be corrected by the treating surgeon. However, this is usually not completely successful. A repositioning of 0° is all that is achieved in many cases. In this case, distal fracture fragment 10 in the sagittal plane according to FIG. 2b can be fixed only normal to the radius shaft 11.

This previously known repositioning takes place by means of a T-shaped osteosynthesis plate 7, in short a T-shaped plate 7, the short and long legs 12, 13 whereof are screwed according to FIG. 2b to fracture fragments 10 and radius shaft 11 by means of bone screws 8 engaging through openings 9 in T-shaped plate 7 in the palmar direction according to reference number 6, radius 1 occupying the position shown in FIG. 2b with respect to radius shaft 11. Bending ankle 5 between short and long leg 12 and 13 respectively amounts to approx. 20° (FIG. 2b).

In order to make it easier for the surgeon to achieve the remaining 10° tilt of the articular surface in the palmar direction according to reference number 6 together with employed T-shaped plate 7, lower face 14 of long, proximal leg 13 facing the proximal bone or radius shaft 11 is provided according to the present invention with a spacer 15 in the form of a wedge 16 in order to keep long leg 13 at a specific angle γ of between 5° and 20° by means of its wedge faces. Wedge 16 can be held in particular by means of a bone screw 17, which passes through holes 18 and 19 in long leg 13 and in wedge 16 (FIG. 4a). When pre-formed T-plate 7 is placed on bone or radius shaft 11, after short leg 12 of plate 7 has already been fastened distally on dislocated fracture fragments 10 by means of bone screws 8, long leg 13 of T-plate 7 does not therefore lie directly on proximal bone or radius shaft 11, but rather stands away by several millimetres, depending on the size of angle γ. An angle γ between 5° and 20° thus results between distal fracture fragment 10 fixed in a neutral position (neutral with respect to the shaft axis of proximal bone or radius shaft 11) and T-plate 7. If wedge 16 is now removed by the surgeon and T-plate 7 is pressed with its long leg 13 on proximal bone or radius shaft 11, distal fracture fragment 10 fastened to short leg 12 of T-shaped plate 7 is tilted through angle γ of previously introduced and now removed wedge 16 in the palmar direction (reference number 6) according to FIG. 3b. The anatomy of distal radius 1 with a 10° palmar tilt in the sagittal plane (FIG. 3b) is thus reproduced in a straightforward manner. Spacer 15, in this case wedge 16, leads to an anatomical repositioning of at least approx. 15°.

Spacer 15 forms a distancing element 20 disposed on lower surface 14 of long leg 13 facing proximal bone or radius shaft 11 near the body. According to FIGS. 3a and 4a, this can be a wedge 16, which is fastened according to FIG. 4a by means of a bone screw 17, which passes through holes 18 and 19 in long leg 13 of T-shaped plate 7 and wedge 16 (FIG. 5a) and which is screwed into proximal bone or radius shaft 11. According to FIG. 5b, however, distancing element 20 can also be a cylinder 22 or according to FIG. Sc a parallelepiped 23, which in each case are provided like wedge 16 with holes 19 for the fixing.

According to FIG. 4b in combination with FIG. 5d or 5e, distancing element 20 can also be a distancing screw 24 with a shank thread 25 or a screw pin 26 with a smooth shank 27, which in each case are provided with contact faces 21 for proximal bone or radius shaft 11 near the body. Distancing screw 24 and screw pin 26 both have a thread 29 on the head 28, with which they can be screwed into a threaded hole 30 in long leg 13 of T-shaped plate 7 and connected thereto.

By means of distancing ,screw 24 or screw pin 26 of differing length, arbitrary distances between long leg 13 of T-shaped plate 7 and proximal bone or radius shaft 11 and therefore arbitrary angles γ can be adjusted. It can thus be seen that a spacer 16 is provided as a distancing element 20, which is not necessarily a solid wedge 16, but rather other geometrical figures can also be used.

The method for the treatment of distal radius fractures by means of T-shaped osteosynthesis plate 7 of the repositioning device described above is characterised in that short, distal leg 13 of T-shaped plate 7 is rigidly screwed to distal fracture fragment 10, that long, proximal leg 13 of T-shaped plate 7 is held spaced apart by means of spacer 15 or distancing element 20 at a specific angle γ between 5° and 20° with respect to proximal bone or radius shaft 11 facing inwards towards the body and that, after removal of spacer 15 or distancing element 20, long leg 13 of T-shaped plate 7 is pressed onto proximal bone or radius shaft 11 and is fastened there by means of bone screws 8, as a result of which distal fracture fragment 10 fastened to T-shaped plate 7 is tilted by adjusted angle γ in the palmar direction according to reference number 6 (FIG. 3a, 3b).

The anatomical adaptation of T-shaped plate 7, which has an angulation or an angle δ of approx. 20° of short leg 12 of T-shaped plate 7 with respect to long leg 13, rules out the possibility of this pre-bending alone also leading automatically to a corresponding fracture repositioning. The 0° position of the articular surface is on the contrary reached with the angular T-shaped osteosynthesis plates previously known in the prior art, as has already been mentioned in the introduction. As a result of the inventive embodiment of angled T-shaped plate 7, said angulation automatically resulting from the plate pre-bending through angle δ and the use of a spacer 15 or distancing element 20, e.g. in the form of wedge 16, the fracture is “forced” into the required tilt. With the angulation of T-shaped plate 7 alone, this is only possible if T-shaped plate 7 somehow “is held in air” during the operation. This is accompanied by the risk of T-shaped plate 7 then not lying orthograde on bone shaft or radius shaft 11, but being displaced laterally. The surgeon cannot possibly bring a T-shaped plate 7 held by hand in the air subsequently up to bone or radius shaft 11 with the required precision. Moreover, a defined spacing is thus also not complied with that subsequently permits a predictable tilt of fracture fragment 10. This purely “manual” precision is not sufficient and is fundamentally changed by the repositioning device according to the invention and the method performed with said repositioning device.

T-shaped osteosynthesis plate 7 alone is not a repositioning device for the treatment of distal radius fractures, but rather a pure support plate, which is adapted by its angulation to the anatomy and is placed on a bone preset by other fixing elements such as Kirschner wires and already prefixed anatomically and, due to its angulation, does not then displace the latter again during the fixing.

There follows a comparison of the chronological sequence in the case of

A: a normal operation with a T-shaped osteosynthesis plate according to the prior art:
1. fracture presentation,
2. setting—i.e. resetting,
3. temporarily holding the repositioning with wires,
4. obtaining a desired fracture position,
5. when the desired fracture position has been reached, fitting of the T-shaped osteosynthesis plate,
6. fixing of the T-shaped plate.
   with

B: the chronological sequence with an osteosynthesis plate in the first embodiment of the invention with a wedge:
1. fracture presentation,
2. setting—i.e. resetting,
3. holding the repositioning with wires,
4. then—irrespective of the obtained repositioning—fitting of the T-shaped plate with the wedge,
5. fixing of the plate with the repositioning wedge on the bone or radius shaft,
6. fixing of the fracture,
7. removal of the wedge and now—by bringing the plate up to the bone—obtaining the desired repositioning.

This comparison of the repositioning procedures according to the prior art and according to the invention shows that the chronological sequence in a normal operation agrees with the chronological sequence with the repositioning device according to the invention only in steps 1 to 3. The sequence with the use of the repositioning device according to the invention then takes, with steps 4 to 7, a different procedural route from the previously known method which then comprises steps 4 to 6.

### LIST OF REFERENCE NUMBERS

1 radius
2 ulna
3 metacarpus
4 metacarpal bone
5 dorsal
6 palmar
7 plate
8 bone screw
9 opening
10 fracture fragment
11 radius shaft
12 short leg
13 long leg
14 lower surface
15 spacer
16 wedge
17 bone screw
18 hole
19 hole
20 distancing element
21 contact face
22 cylinder
23 parallelepiped
24 distancing screw
25 shank thread
26 screw pin
27 shank
28 head
29 thread
30 threaded hole

1. A repositioning device for the treatment of distal radius fractures by means of a T-shaped osteosynthesis plate, with a long leg and a short leg disposed T-shaped and at an angle with respect to said long leg, wherein the short, distal leg has a surface coming into contact with the distal fracture fragment and a plurality of openings for bone screws and the long, proximal leg is intended to come into contact with the proximal bone near the body, characterised in that the lower surface of the long, proximal leg of the T-shaped plate facing the proximal bone or radius shaft is provided with a spacer in the form of a wedge in order to keep the long leg at a specific angle (γ) to the proximal bone near the body.

2. The repositioning device according to claim 1, characterised in that the wedge is disposed on the lower surface of the long leg facing the proximal bone near the body.

3. The repositioning device according to claim 1, characterised in that the wedge forms an angle (γ) of 5 to 20° between its wedge faces resting on the proximal bone near the body and the lower surface of the long leg facing said proximal bone.

4. The repositioning device according to claim 1, characterised in that the wedge is fastened detachably on the lower surface of the long leg facing the proximal bone near the body.

5. A repositioning device for the treatment of distal radius fractures by means of a T-shaped osteosynthesis plate, with a long leg and a short leg disposed T-shaped and at an angle with respect to said long leg, wherein the short, distal leg has a surface coming into contact with the distal fracture fragment and a plurality of openings for bone screws and the long, proximal leg is intended to come into contact with the proximal bone near the body, characterised in that the lower surface of the long, proximal leg of the T-shaped plate facing the proximal bone or radius shaft is provided with a spacer in the form of a distancing screw or a screw pin with a contact face for the proximal bone, said distancing screw or screw pin being able to be screwed in the long leg, in order to keep the long leg at an angle (j) to the proximal bone near the body.

6. The repositioning device according to claim 5, characterised in that the distancing screw has a head thread and a smooth shank.

7. The repositioning device according to claim 2 characterised in that the wedge forms an angle (γ) of 5 to 20° between its wedge faces resting on the proximal bone near the body and the lower surface of the long leg facing said proximal bone.

8. The repositioning device according to claim 2 characterised in that the wedge is fastened detachably on the lower surface of the long leg facing the proximal bone near the body.

9. The repositioning device according to claim 3 characterised in that the wedge is fastened detachably on the lower surface of the long leg facing the proximal bone near the body.

10. The repositioning device according to claim 7 characterised in that the wedge is fastened detachably on the lower surface of the long leg facing the proximal bone near the body.
